(19) United States
(12) Patent Application Publication
(10) Pub. No.: US 2007/0083184 A1
(43) Pub. Date: Apr. 12, 2007

(54) Epidural catheter system and methods of use

(76) Inventor: Robert C. Simpson, Tallahassee, FL (US)

Correspondence Address: LOCKE LIDDELL & SAPP LLP, 600 TRAVIS, 3400 CHASE TOWER, HOUSTON, TX, 77002-3095, US

(21) Appl. No.: 11/519,258

(22) Filed: Sep. 11, 2006

Related U.S. Application Data
(63) Continuation-in-part of application No. 09/989,342, filed on Nov. 20, 2001, now abandoned.

(51) Int. Cl. A61M 31/00

(52) U.S. Cl. 604/500

(57) ABSTRACT

An epidural injection is used in medical procedure to administer medication to a patient's epidural space in the spine, usually to alleviate pain. Although effective in purpose, current medical procedure to administer an epidural injection does contain a flaw that exposes the patient to possible infection, usually manifested as an epidural abscess or bacterial meningitis. A source for infection stems from the manner the epidural catheter, specifically the proximal end not being inserted into the patient, is traditionally handled throughout the procedure—usually freely hanging, susceptible to breaking the sterile field and becoming contaminated. The current invention, an epidural catheter dispenser system, seeks to eliminate this risk of epidural catheter contamination by maintaining the epidural catheter, especially the proximal catheter end, in a sterile dispenser that can be easily manipulated by a physician. The epidural catheter dispenser system defines an inner cavity in which an epidural catheter may be loaded. When ready for use, a distal catheter end is extracted from the dispenser's inner cavity through a dispenser aperture on the dispenser's distal end piece, or top, allowing the physician to direct the epidural catheter into an epidural needle bore and into a patient's epidural space. Because the epidural catheter dispenser system and its epidural catheter contents fit easily into the palm of a physician's hand, the proximal catheter end is permanently in a controlled, contained sterile environment throughout the entire catheter placement procedure until extracted from the dispenser. The current invention minimizes and virtually eliminates the risk of epidural catheter contamination. Thus, the epidural catheter dispenser system provides benefits beyond existing epidural injection procedures including: (1) reduced risk of infection of the patient receiving an epidural injection; (2) easier catheter management for the physician; (3) better control of the medical microenvironment for the physician; and (4) improved medical efficiencies.

### BACKGROUND OF THE INVENTION

An epidural injection is a medical anesthetic technique whereby medication, typically an anesthetic agent with or without a steroidal component, is administered to a patient's spine, specifically in the epidural space. The epidural space consists of the space between a patient's bony spinal vertebrae and the dura mater, or tough outer layer of the spinal cord. Epidural injections are most commonly used to alleviate pain associated with childbirth or nerve root impingement. Nerve roots are bands of nerves extending from the spinal cord in the vertebral canal to the body through intervertebral foramina, or spaces between vertebrae. Nerve roots can become impinged as a result of a bulging, herniated or ruptured intervertebral disc due to compression or wear. Nerve root impingement can also occur as a result of a radiculopathy, bony osteophyte or projection compressing the nerve root.

For the administration of an epidural injection, a physician will request the patient to lie in a fetal position, whereby the patient's knees are pulled closely to her chest. This creates a convex curvature for the patient's spine, resulting in the widening of the intervertebral space between each vertebra's spinous process. The physician may then palpate the intervertebral space at the level of the spine in which the epidural injection is desired to be administered to alleviate pain.

After locating the precise point to administer the epidural injection, the physician will use an aseptic technique to prep and drape the area and cleanse the area of bacteria to prevent infection. During the prep and drape process, the physician will use iodine and a brush to scrub the skin surface several times. The physician will also drape the local area with sterile cloths, leaving only the small area to administer the epidural uncovered.

To administer the actual epidural injection the physician will use a sterile, pre-packaged epidural kit containing an epidural needle with a proximal end containing the handle and a distal end that is inserted into the patient. Within the epidural needle's hollow bore is a plunger with a proximal end containing a handle and a distal end that extends into the needle. The epidural needle with its corresponding plunger can vary in length and gauge, or diameter of the hollow bore, depending on the size of the patient. The sterile epidural kit also contains coiled, sterile, flexible catheter tubing, which at the appropriate time is inserted into the epidural needle and directed into the patient's epidural space at various lengths to carry the anesthetic medication into the epidural location.

When administering the epidural injection, the physician, with sterile gloves, will use a local anesthetic, such as lidocaine, on the skin and surface tissue to prevent surface pain from the injection. The physician will then insert the epidural needle into the patient to the epidural space. The physician will then slowly withdraw the plunger in the epidural needle, observing for any backflash. Bloody backflash may indicate the puncture of a vein, or a pulsating backflash of blood may indicate the puncture of an artery. In either scenario, the practitioner may have to readjust the epidural needle and monitor the backflash after removing the plunger.

With satisfactory placement of the epidural needle and removal of the intra-needle plunger, the physician will then remove the sterile catheter tubing from the pre-packaged epidural kit and slowly insert one end of the catheter (also known as the catheter's distal end) into the hollow bore of the needle positioned in the patient. The catheter is then advanced in the needle into the patient until the desired length of catheter insertion is achieved. At this point, to remove the epidural needle and leave only the catheter in the patient, the physician firmly secures the catheter in the patient with one hand, while simultaneously withdrawing the needle away from the patient with the other hand. With the withdrawal of the needle, the entire catheter passes through the hollow bore of the epidural needle. When the needle has been successfully removed from the patient through the entire length of the catheter tubing, the proximal end of the catheter (e.g. the catheter end not inside the patient) is ready to receive the medication for introduction into the patient. The catheter can be immediately withdrawn from the patient after medication is administered, or the catheter can be left in the patient as an in-dwelling catheter for extended periods of medication administration.

As a modestly invasive procedure, an epidural injection can have adverse effects, one of which is infection. Infection from an epidural procedure can manifest as an epidural abscess or, in severe cases, bacterial meningitis, an infection of the meninges of the central nervous system. Curative measures for an epidural abscess can require surgical drainage, while bacterial meningitis may require extensive antibiotics and significant hospitalization.

Ruling out blood-borne sources of infection from the patient, a primary source of infection for an epidural injection is manifest in the procedure itself. A contributing factor influencing the likelihood of infection from the procedure is the narrow sterile field in which the procedure occurs, as defined by the area on the patient that the physician prepped and draped. Any contaminants outside this narrow sterile field can potentially enter the field and provide a source for infection. The most significant factor contributing to the likelihood of infection from an epidural injection is the free, proximal end of the catheter that is dangling freely as the catheter's distal end is inserted into the epidural needle and into the patient. Because the catheter is packaged in a coiled form, upon unraveling for use, the lengthy catheter tubing is very difficult to manage and control as it is being inserted into the patient's epidural space. The difficulty in controlling the free end of the catheter is further compounded by the inability of the physician to utilize both hands to manipulate the catheter. Because the practitioner must keep one hand holding the needle in the patient, the practitioner can only utilize the other hand to control the catheter tubing's free proximal end while simultaneously trying to maneuver the distal, inserted end of the catheter. Furthermore, during the needle removal process after the catheter has been introduced into the patient, the needle must be removed while passing the entire catheter through the needle's bore which poses additional opportunity to lose control of the catheter. The practitioner is limited in the ability to keep the catheter inside the patient, remove the needle and control the free-hanging portion of the catheter.

In consideration of these factors involved during the procedure, the risk of the catheter springing out of the physician's hand can be high and the catheter may subsequently contact areas outside the sterile field, thus breaking the narrow, sterile field and potentially introducing contaminants. In the event control of the catheter is lost and the catheter has become contaminated, prior to insertion, the practitioner must discard the contaminated catheter and re-introduce a new, sterile catheter. In the event the catheter has been inserted into the patient and the distal end breaches the sterile filed, the practitioner must either wipe down the compromised end with alcohol or sever the affected end from the catheter before continuing with the procedure. In this likely scenario, many negative outcomes are produced, including: (1) wasted medical resources in time, supplies and money; (2) additional likelihood of patient infection due to the increased time the patient's closed body system is exposed to the environment; and (3) the loss of the practitioner's ability to respond and control the medical microenvironment while having both hands occupied.

The medical necessity of administering epidural medicaments to alleviate pain or deliver other beneficial medications to the epidural space remains. However, in light of the inherent faults of the epidural injection procedure for potential patient infections, there exists no replacement procedure to eliminate the infection potential, specifically addressing the lack of control over the free-hanging, proximal end of the catheter. Additionally, no current medical device technology attempts to eliminate the lack of control over the free-hanging, proximal end of the catheter to address the issue of potential infection for epidural injections and catheter insertion. Thus, there exists in the medical procedure for epidural injections a need to eliminate the inability of the physician to control the proximal, free-hanging end of the catheter tubing to avoid contamination during insertion of the catheter in the needle bore and during the removal of the needle from the patient's body while pulling the catheter through the needle's bore.

### SUMMARY OF THE INVENTION

The epidural catheter dispenser system of the current invention addresses the shortcomings of current medical practice to prevent infection during the epidural injection procedure. The current invention's dispenser system includes a cylindrical dispenser with a rounded, conical top, all of which is made from rigid or semi-rigid material, most preferably plastic. The conical top of the current invention's dispenser contains a small aperture that provides a conduit between the dispenser's inner cavity and the casing's exterior. On the bottom of the current invention's dispenser, a larger aperture exists that provides a conduit between the dispenser's inner cavity and the casing's exterior.

The current invention's dispenser surrounds an inner cavity, providing a hollow interior for the epidural catheter dispenser system. The system of the current invention involves the “loading” of an epidural catheter of variable length coiled inside the current invention's inner cavity through the aperture on the dispenser's bottom side. With the catheter's placement in the inner cavity of the current invention, a distal end of the catheter is threaded through the aperture on the dispenser's conical top, thus leaving the proximal end of the catheter in a controlled, contained environment. The epidural catheter dispenser system of the current invention is now prepared for use by a physician.

In practical usage for epidural injections, the current invention's epidural catheter dispenser system is placed in either one of the physician's hands after the epidural needle has been satisfactorily placed in the patient's back. With one hand holding the epidural needle in place and the other hand holding the epidural catheter dispenser system, the physician will cup the current invention's dispenser with the third, fourth, and fifth digits of her hand, leaving the thumb and index finger free to manipulate the catheter. Grasping the distal end of the coiled catheter, which was previously threaded through the aperture on the dispenser's conical top from the inner cavity, with her free thumb and index finger, the physician then directs the catheter's distal end to the central bore of the epidural needle. With successful introduction of the catheter into the epidural needle, the physician can manually advance the catheter to the desired length into the epidural needle and into the patient with her thumb and index finger while simultaneously controlling the epidural catheter dispensing system in the palm of the same hand with her third, fourth, and fifth digits.

After the catheter has been advanced from the epidural catheter dispensing system of the current invention to the desired length inside the patient's epidural space, the physician may remove the epidural needle safely and effectively using the epidural catheter dispensing system. To remove the epidural needle from the patient, the epidural needle is withdrawn by the practitioner's hand not holding the dispenser system of the current invention. Once the epidural needle has been completely withdrawn from the patient's skin and soft tissue, the base of the needle is transferred to the thumb and index finger, or alternatively to the index finger and the third digit, of the practitioner's hand holding the epidural catheter dispensing system of the current invention. The practitioner's hand originally holding the epidural needle is now transferred to hold the area of the catheter immediately adjacent to the patient's skin where the catheter enters the patient's back. Securing the catheter firmly next to the patient's skin, the practitioner can completely withdraw the epidural needle with the catheter running through the needle's bore by simultaneously pulling the epidural needle and the epidural catheter dispensing system of the current invention back with the same hand such that the epidural needle runs the entire length of the remaining catheter in the inner cavity of the current invention's dispenser up to the catheter's proximal end. Thus, during the needle withdrawal procedure, the physician—with one hand—would have maneuvered the entire length of catheter through the epidural needle's bore.

Concerning the entire procedure of administering an epidural injection, the unique advantages of the epidural catheter dispensing system are appropriate and desirable. By placing the catheter's proximal end in a controlled, closed and sterile dispenser, the epidural catheter dispensing system of the current invention eliminates the catheter's freely-hanging proximal end in traditional procedures, thus eliminating any likelihood of contaminating the catheter as a source for possible infection. Without having to contend with a freely-hanging proximal end of a catheter, any physician using the current invention's epidural catheter dispensing system can better command and maneuver the catheter and epidural needle during the procedure to maintain control of the medical environment. Additionally, the physician does not have to concern herself with the possibility of the catheter breaking the sterile field, thus allowing the practitioner to concentrate more fully on the patient and the epidural injection procedure.

The current invention's epidural catheter dispensing system is also socioeconomically desirable. Because the current invention's epidural catheter dispensing system offers a reduced likelihood of infection risk from epidural injections, patient satisfaction and morbidity will likely improve following such procedure; and therefore, the amount of medical malpractice litigation stemming from or involving such procedure will likely decrease proportionately. Extending this societal benefit further, from a products liability standpoint, manufacturers of epidural products and catheters would likely experience decreased liability with the addition of the epidural catheter dispensing system of the current invention because of the benefits in infection control. Alternatively, patients who are subjected to an epidural injection with a catheter not having the current invention's epidural catheter dispensing system and who experience ill effects from such procedure, specifically infection, may recover damages from manufacturers who are aware of the current invention's dispensing system but who choose not to include the current invention in their epidural injection products package. Thus, the current invention's epidural catheter dispensing system is desirable for patients and product manufacturers.

No current medical technology has addressed the problems with catheter contamination contributing to infection inherent in current epidural injection procedure, especially in a handheld device or system sterilely containing the catheter that the physician may manipulate in one hand. A technology system does exist for a handheld storage system for guidewires used in catheter exchange procedures primarily in cardiologic and angioplastic procedures; however, no similar technology has been adapted for the use of catheters in epidural injection procedures. That the handheld guidewire technology has not been adapted for the use of epidural catheters in epidural injection procedures illustrates the difference in application and technologies between a handheld guidewire storage system and the epidural catheter dispenser system of the current invention. For example, both technologies are used in vastly different procedures —the guidewire is used primarily as an internal placement and guidance device for catheters inside patients and used in procedures where the physician many insert and withdraw many different catheters of varying gauges or configurations from the patient (a procedure known as catheter exchange). Contrastingly, catheters are used as the primary conduit into a patient's body to administer medications or take internal body readings. Thus, catheters and guidewires functionally serve vastly different medical purposes, and no current medical device exists addressing the prevention of catheter contamination in a handheld device.

Catheters and guidewires characteristically differ such that the adaptation of the handheld guidewire storage device to the epidural catheter for epidural injections would be impractical. Guidewires used in cardiologic and catheter exchange procedures vary in length from 190-400 cm and are made of a solid rigid metallic material, likely stainless steel. Because guidewires are solid and made of a rigid metallic material, the guidewires' elastic properties are resilient; however, the guidewires' thin gauge to navigate the human vasculature makes the guidewires susceptible to kinking. In contrast, epidural catheters are hollow tubes of impermeable plastic typically less than 100 cm in length. The plastic properties allow the epidural catheter's elasticity to be extremely flexible; and the epidural catheter's hollow gauge, larger than that of the solid guidewire, makes the epidural catheter less susceptible to kinking. Thus, in light of the differences between guidewire properties and epidural catheter properties, a handheld storage device for guidewires would be impractical for use with epidural catheters without improvements and adaptations in the device as applied to use with the epidural injection procedure intended and the epidural catheter materials used.

### BRIEF DESCRIPTION OF THE DRAWINGS

FIG. 1A is a lateral view of the epidural catheter dispenser system with an epidural catheter.

FIG. 1B is an aerial view of the epidural catheter dispenser system with an epidural catheter.

FIG. 1C is a lateral view of the epidural catheter dispenser system without an epidural catheter.

FIG. 1D is an angled view of the epidural catheter dispenser system without an epidural catheter.

FIG. 2 is an epidural needle.

FIG. 3 illustrates proper handling and positioning of an epidural needle.

FIG. 4 illustrates the insertion of an epidural needle into a patient.

FIG. 5 illustrates the introduction of the epidural catheter dispenser system to the epidural injection procedure.

FIG. 6 illustrates the insertion of the distal epidural catheter end into the epidural needle bore.

FIG. 7 illustrates the successful insertion of the distal epidural catheter end into the patient's epidural space.

FIG. 8 illustrates early-staged withdrawal of the epidural needle from the patient.

FIG. 9 illustrates the continued removal of the epidural needle from the patient.

FIG. 10 illustrates the completed removal of the epidural needle from the patient and successful completion of the epidural catheter placement in the patient's epidural space.

### DESCRIPTION OF ILLUSTRATIVE EMBODIMENTS

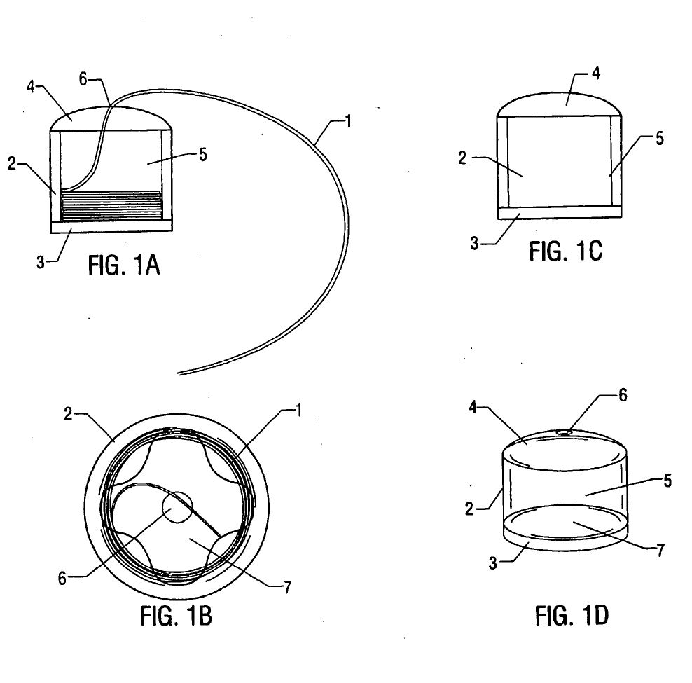

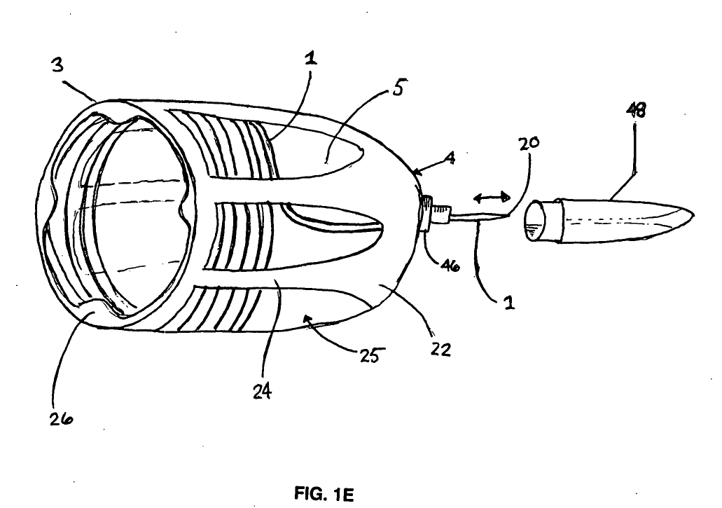

FIG. 1A illustrates a lateral view of a preferred embodiment for the epidural catheter dispenser system with an epidural catheter. The catheter tubing 1 is shown with a portion of the catheter outside the dispenser and with the remaining portion of the catheter wound inside the dispenser's inner cavity 5. Preferably, and to prevent tangling, the catheter is wound flat against the bottom of the dispenser. The dispenser's inner cavity 5 is defined in the preferred embodiment by the sidewall 2 which can take a cylindrical shape, as illustrated, or a conical or other polyhedral shape. In some embodiments, the dispenser may have a spherical or cubic shape. The proximal end piece 3 defines the bottom border of the inner cavity 5 and the distal end piece 4 defines the upper border of the inner cavity. While not visible from the lateral view of FIG. 1A, a dispenser aperture 6 on the distal end piece 4 allows the catheter 1 to pass through from the inner cavity 5 to the exterior environment.

FIG. 1B illustrates an aerial view of the preferred embodiment for the epidural catheter dispenser system. The catheter 1 is again illustrated as wound in the dispenser's inner cavity, as defined by the sidewall 2. The catheter exits the inner cavity via the dispenser aperture 6 on the distal end piece of the current invention's preferred embodiment. A loading aperture 7 located on the proximal end piece allows for the loading or adjusting of the catheter into the inner cavity of the dispenser.

FIG. 1C illustrates a lateral view of the preferred embodiment of the epidural catheter dispenser system without the loaded epidural catheter. There exists in the preferred embodiment a sidewall 2 connected to a proximal end piece 3 which forms the dispenser's bottom, and a distal end piece 4 which forms the dispenser's top. The sidewall and proximal and distal end pieces define an inner cavity 5 where the sterile epidural catheter is loaded for use. The sterile inner cavity 5 maintains the sterile environment for which the epidural catheter is contained during the epidural catheter placement in an epidural injection procedure, reducing the likelihood of catheter contamination and patient infection.

FIG. 1D illustrates an angled view of the epidural catheter dispenser system's preferred embodiment without a loaded epidural catheter. The dispenser's preferred embodiment includes a sidewall 2 connected to a proximal and a distal end piece 3 and 4, respectively, thus forming a sterile inner cavity 5 where the epidural catheter is contained. Although the sidewall is illustrated taking a cylindrical form, the sidewall may also include other forms such as a conical shape or other polyhedral shape. An epidural catheter is loaded into the dispenser's inner cavity through a loading aperture 7 located and defined on the proximal end piece 3. During the epidural catheter placement procedure in a patient, the catheter is extracted from the inner cavity 5 for use through the dispenser aperture 6 located and defined on the distal end piece 4. Because the sterile epidural catheter is controlled and contained in a sterile environment throughout the epidural catheter placement portion of the procedure, the preferred embodiment of the current invention reduces the likelihood of catheter contamination and possible patient infection.

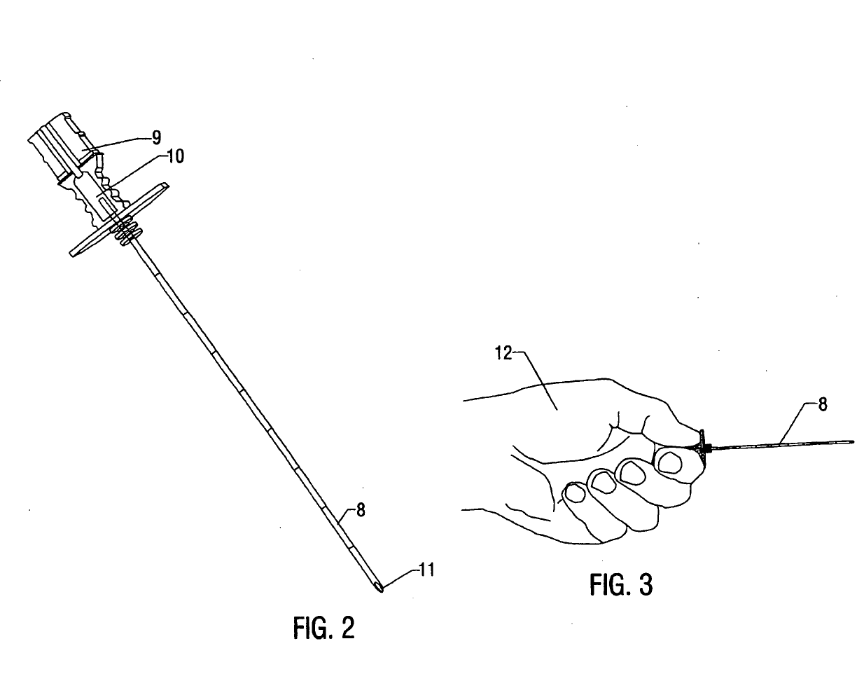

FIG. 2 illustrates an epidural needle 8 which although not part of the preferred embodiment of the current invention, serves to illustrate the preferred use of the epidural catheter dispensing system of the current invention. The distal end of the epidural needle is the tip, which is inserted into the patient to the patient's epidural space. The proximal end of the epidural needle 10 is the needle's handle the physician uses to control the epidural needle. Coursing the entire length of the epidural needle 8 from the handle to the needle tip is a hollow bore 11 through which the needle plunger 9 is inserted as well as the epidural catheter once the needle plunger 9 has been removed during an epidural injection procedure. The needle plunger 9 is utilized during the epidural procedure to determine whether the physician has punctured any vasculature. By withdrawing the needle plunger 9 and observing the coloration of any backflash fluids coming through the epidural needle bore 11 the physician can better determine the location of the distal end of the needle inside the patient. With satisfactory placement of the epidural needle 8 the physician will completely withdraw the needle plunger, 9, from the epidural needle bore 11 allowing for the insertion of the epidural catheter. FIG. 3 illustrates the proper handling of an epidural needle 8 in the physician's hand 12. The epidural needle 8 can be used in either of the physician's hands, but as illustrated, the epidural needle 8 is in the physician's left hand 12. The proper handling of the epidural needle 8 is such that the physician has the epidural needle handle positioned between the thumb and index finger for maximum control and dexterity in maneuvering the needle to the appropriate position on the and in the patient.

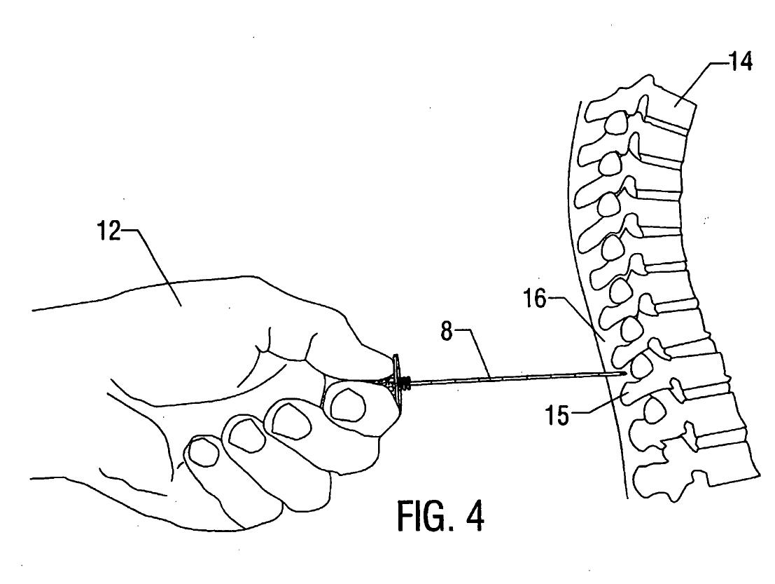

FIGS. 4 through 10 illustrate the preferred method of use for the preferred embodiment of the current invention's epidural catheter dispenser system and how the current invention is used to place an epidural catheter in conducting an epidural injection procedure. Referring to FIG. 4, the illustration shows the introduction of the epidural needle 8 into the patient's body 14 specifically at the location between the vertebral spinous processes 15 also known as the intervertebral space 16. The physician inserts the epidural needle 8 into the patient 14 until the distal end of the needle has reached the epidural space. With satisfactory placement of the epidural needle 8 the physician will withdraw the needle plunger completely if no bloody flashback is observed through the epidural needle bore. The physician's left hand 12 is illustrated properly manipulating the epidural needle 8 between the thumb and index finger.

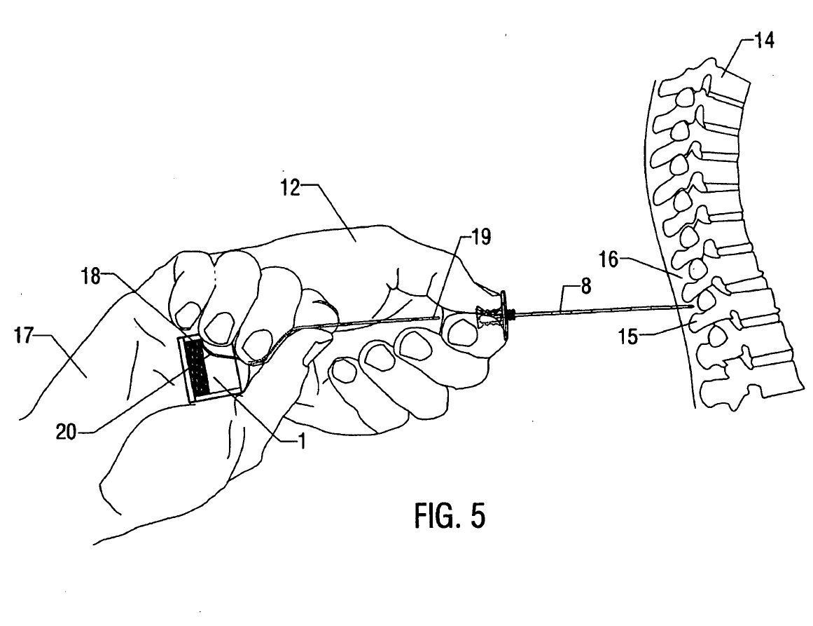

FIG. 5 illustrates the introduction of the epidural catheter dispensing system of the current invention to the epidural injection procedure. The physician has already successfully placed the epidural needle 8 into the patient 14 at the desired location. The physician has also completely removed the needle plunger from the epidural needle bore. The epidural needle 8 is now prepared to receive the epidural catheter 1 for introduction into the patient's epidural space. As FIG. 5 illustrates, the physician, while holding the epidural needle 8 in the patient's back with the left hand 12 has now grasped the epidural catheter dispensing system 18 into the right hand 17. The dispenser 18 is positioned in the physician's righthand palm 17 and is secured with the third, fourth, and fifth digits. The physician has also grasped the distal catheter end 19 with the thumb and index fingers of the right hand and has positioned the distal catheter end 19 for insertion into the epidural needle bore. The remainder of the epidural catheter 1 and the proximal end of the catheter 20 stay contained and controlled in the sterile environment of the dispenser's inner cavity, avoiding any possibility of contamination.

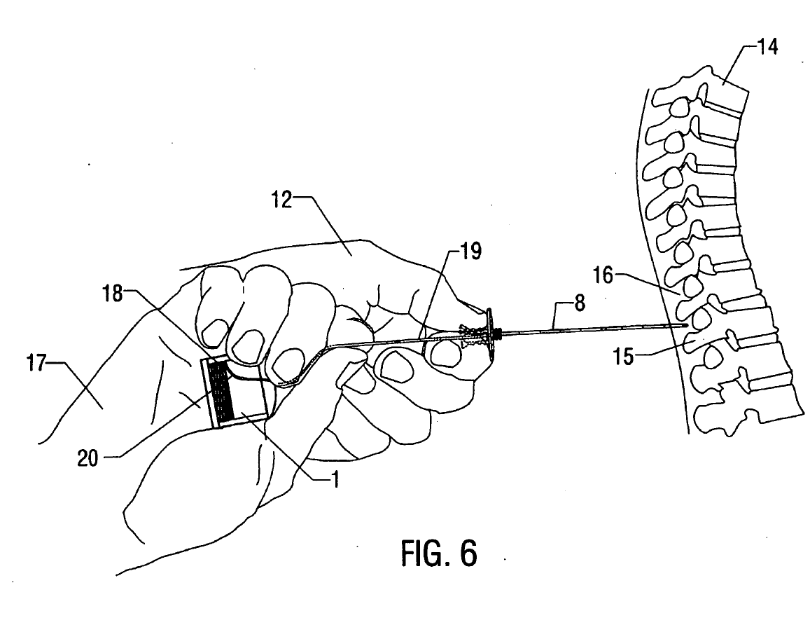

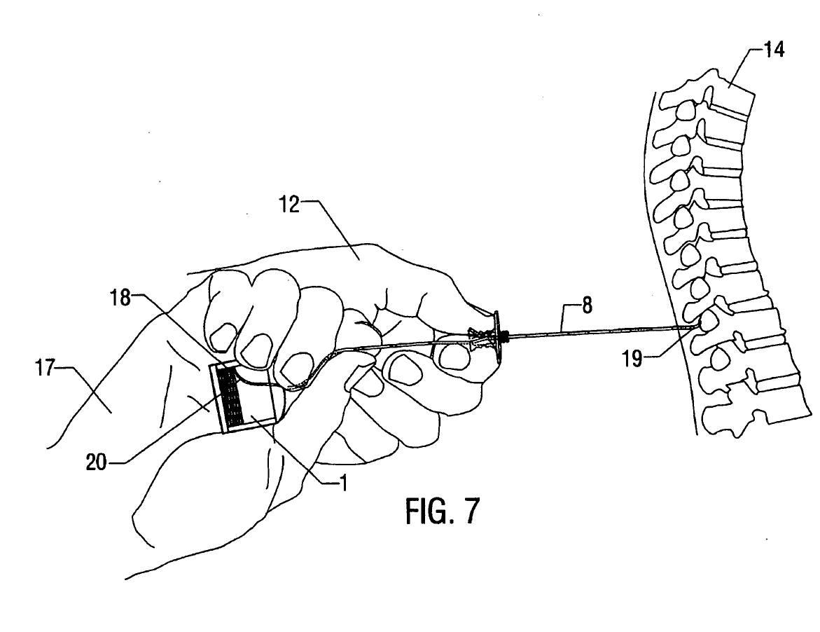

FIG. 6 illustrates the successful insertion of the distal epidural catheter end 19 into the epidural needle bore. The physician is still maintaining control of the epidural needle 8 in the patient 14 with the thumb and index finger of the left hand 12. The physician still has control over the epidural catheter dispensing system 18 in the palm of the right hand 17 secured with the third, fourth, and fifth digits. As illustrated, the epidural catheter 1 and its proximal end are clearly confined in the inner cavity 5 of the epidural catheter dispenser system such that there is no likelihood for the catheter to become contaminated as a possible source of infection. The physician at the stage illustrated in FIG. 6 will advance the distal epidural catheter end 19 the entire length of the epidural needle 8 into the patient's epidural space.

FIG. 7 illustrates that the physician has utilized the epidural catheter dispensing system 18 to successfully introduce the distal catheter end 19 into the patient's body and epidural space 14. Although the distal catheter end 19 is in the patient, a portion of the epidural catheter 1 is in the epidural needle 13 bore with the remainder, including the proximal catheter end, still in the epidural catheter dispensing system 18, specifically in the inner cavity 5. The physician's left hand 12 is still controlling the epidural needle, and the physician's right hand 17 is singly controlling the dispenser 18 with the epidural catheter 1. Throughout the epidural injection procedure to this point, the epidural catheter 1 and its proximal end 20 have remained confined in the dispenser's sterile inner cavity. As the catheter is in a controlled, contained dispenser 18 the likelihood for contamination of the catheter is eliminated.

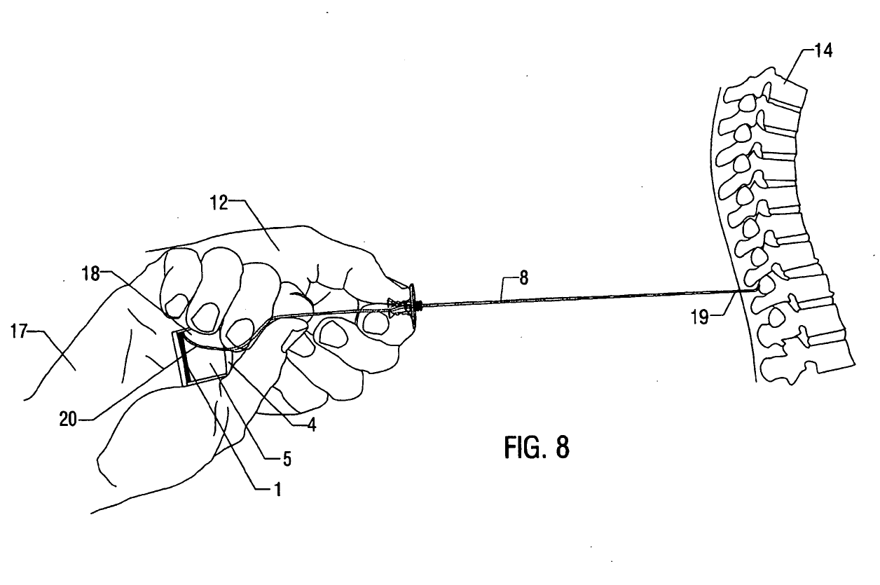

FIG. 8 illustrates the early-staged withdrawal of the epidural needle 8 from the patient 14 once the distal catheter end 19 has been successfully placed into the patient's epidural space. The physician is still maintaining control of the epidural needle 8 with the thumb and index finger of the left hand 12; however, the physician has now pulled the epidural needle 8 from the patient, leaving the distal catheter end 19 in the patient. The epidural catheter 1 still traverses through the epidural needle bore, and the physician must completely pull the epidural catheter 1 through the epidural needle bore to totally remove the epidural needle. Through this process, the physician with the right hand, palm, and third, fourth, and fifth digits 17 is still maintaining control of the proximal end of the epidural catheter 20 inside the inner cavity 5 of the epidural catheter dispenser 18. FIG. 8 illustrates that during this procedure, most of the epidural catheter 1 has been extracted from the inner cavity 5 of the dispenser through the dispenser aperture defined by the dispenser's distal end piece 4.

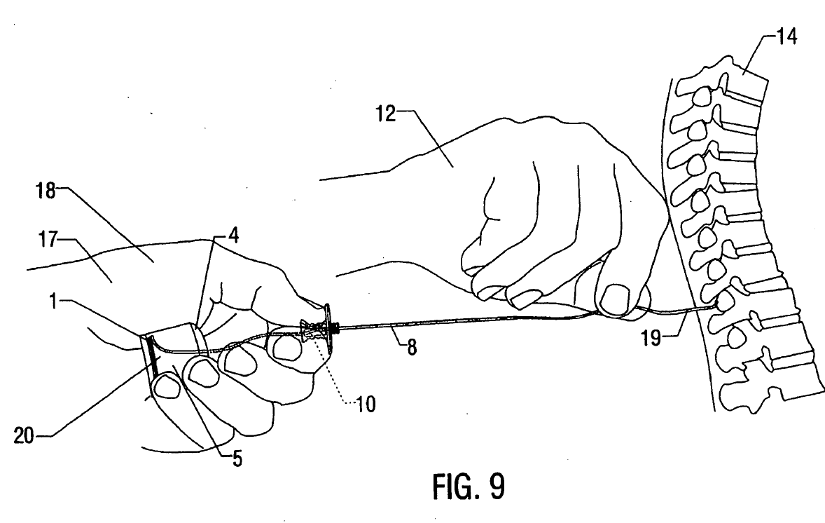

FIG. 9 illustrates the continued removal of the epidural needle 8 from the patient 14. In traditional epidural injection procedure, the physician must contend with contamination of the proximal catheter end during the epidural needle removal process. In contrast, the physician utilizing the preferred embodiment of the current invention's dispenser 18 can withdraw the epidural needle 8 and pull the epidural catheter 1 through the epidural needle's bore with one hand, thereby keeping the catheter inside the dispenser's inner cavity and preventing catheter contamination. In this illustration, the physician is still utilizing the right hand 17 controlling the epidural catheter dispenser system 18 with righthand palm and third, fourth and fifth digits. Simultaneously, the physician's righthand thumb and index finger, or alternatively the right index finger and third digit (not illustrated), grasps the proximal handle on the epidural needle 10. The physician's left hand 12 has now moved to grasp the distal catheter end 19 close to the patient 14 to keep the epidural catheter 1 positioned in the patient. Thus, by firmly gripping the distal catheter end 19 next to the patient 14 while simultaneously pulling back toward the physician with the right hand 17 controlling the dispenser 18 and the epidural needle 8, the physician will extract the remaining epidural catheter 1 from the dispenser's inner cavity 5. By pulling the epidural catheter 1 through the dispenser aperture in the distal end piece 4, and through the epidural needle's bore in one motion, the physician is maintaining the sterility of the epidural catheter 1 especially the proximal catheter end 20 through the epidural needle 8 removal procedure. Throughout the entire epidural needle removal, the proximal catheter end 20 has remained positioned in the dispenser's sterile inner cavity, eliminating all likelihood of catheter contamination.

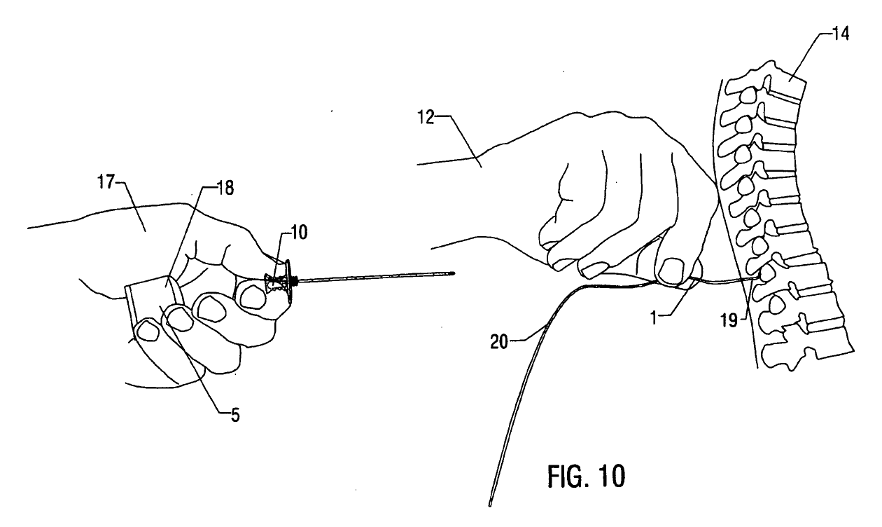

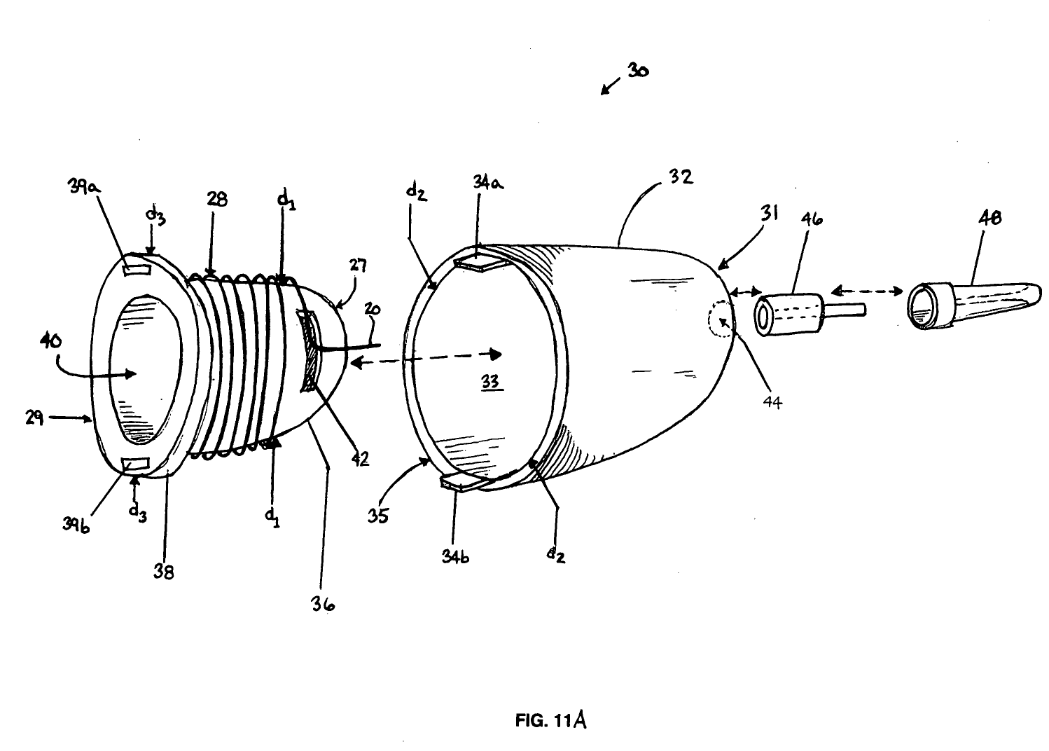

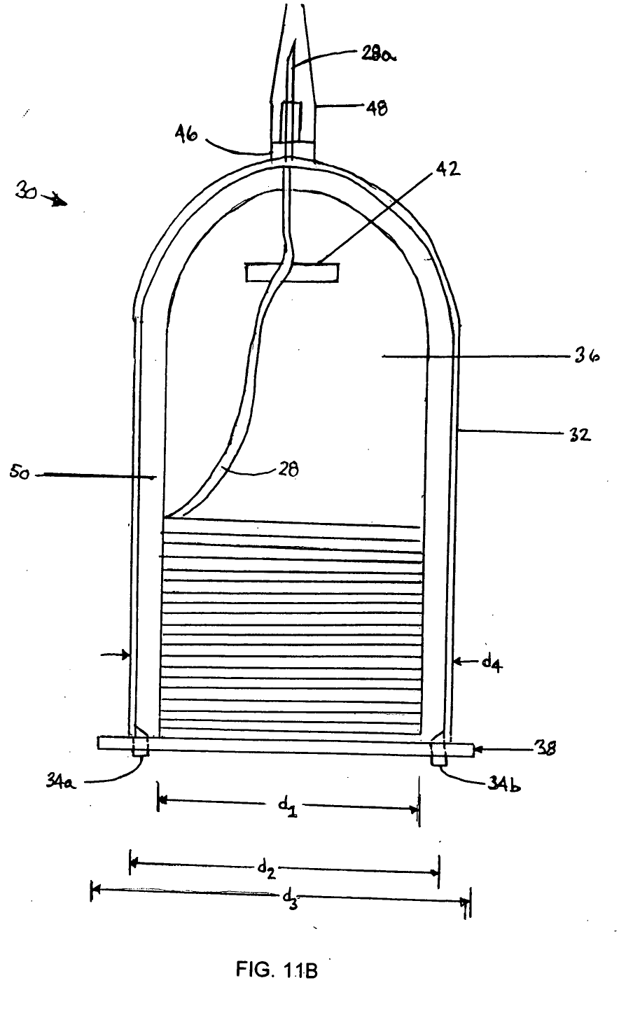

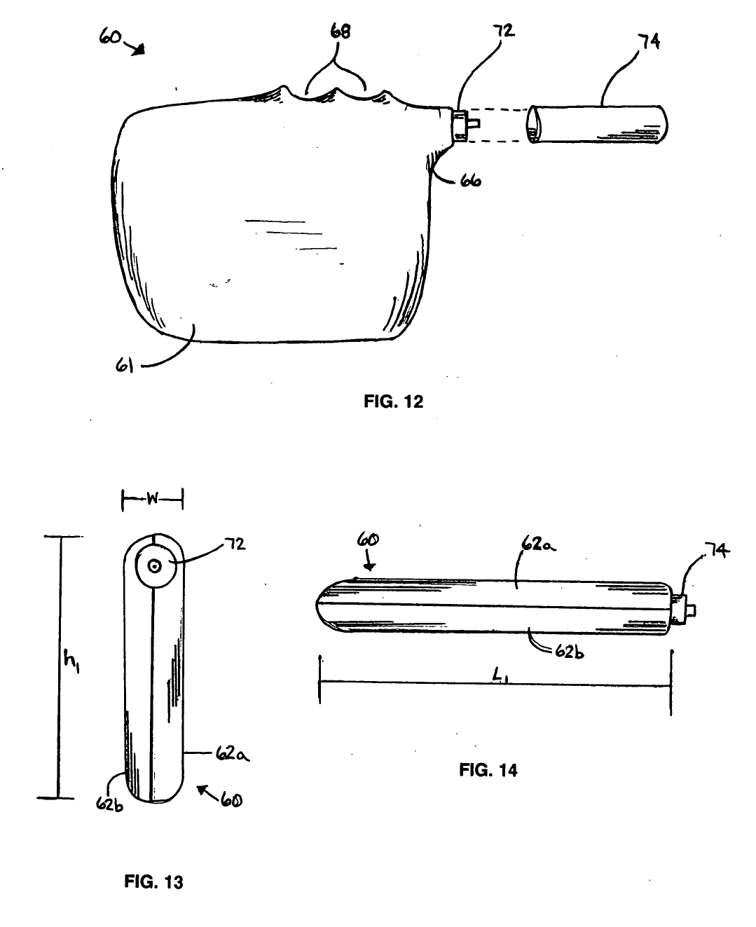

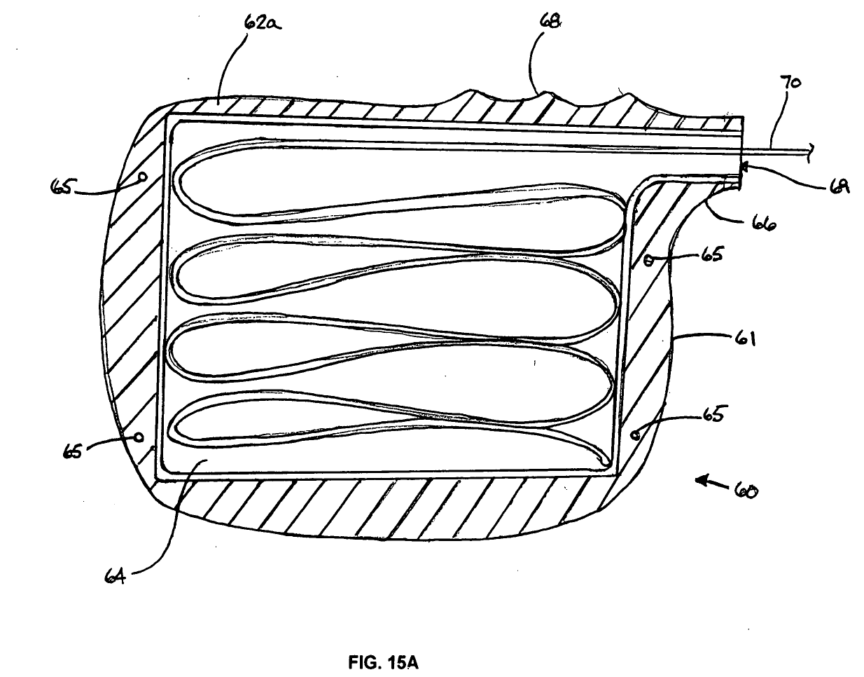

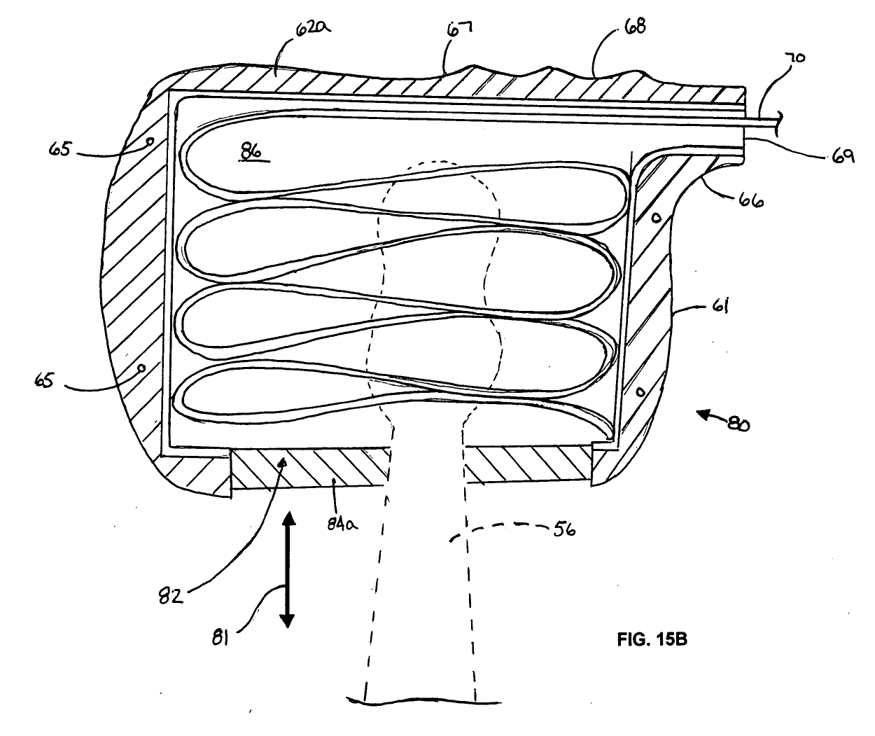

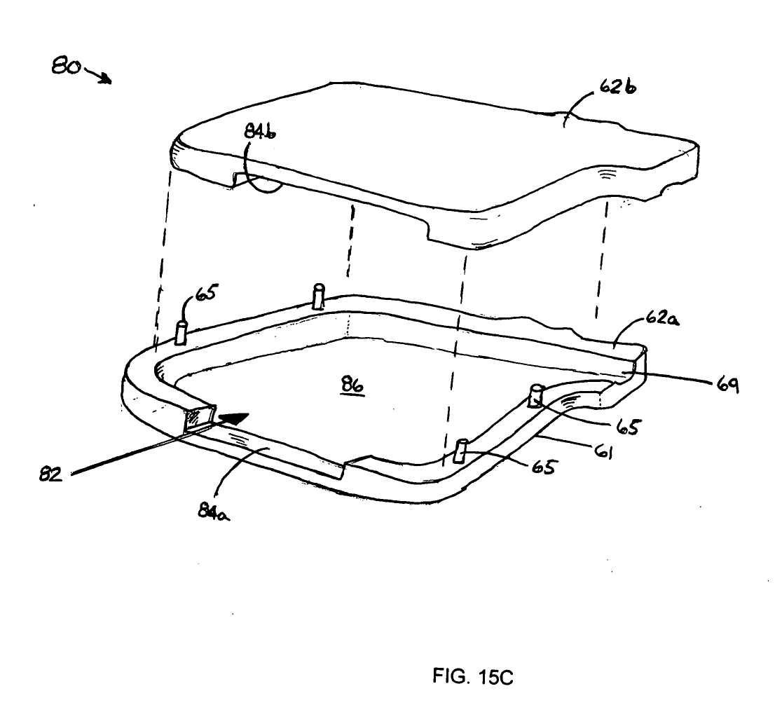

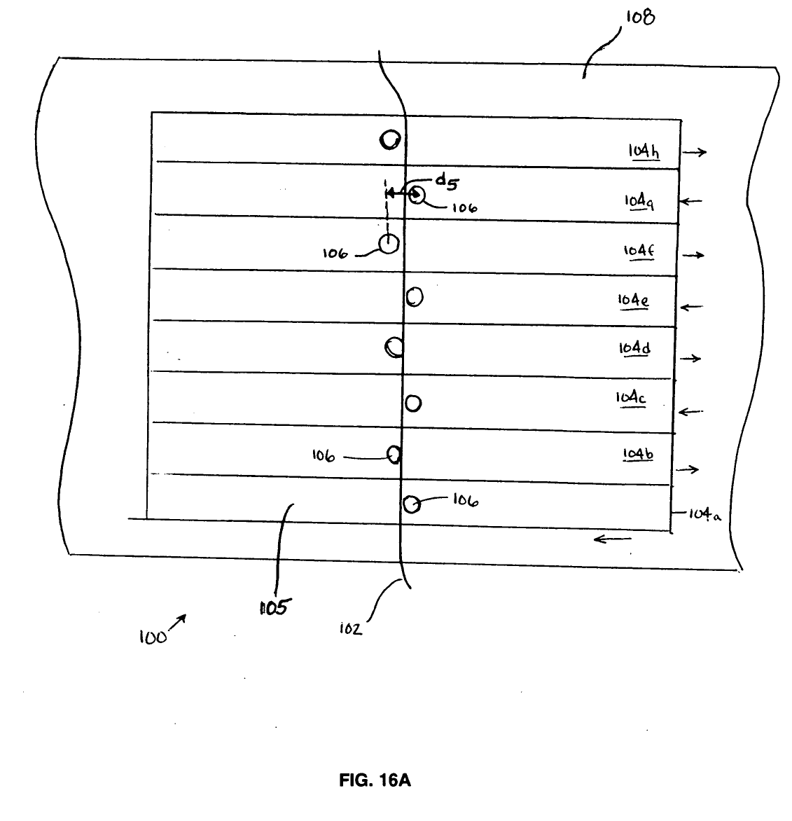

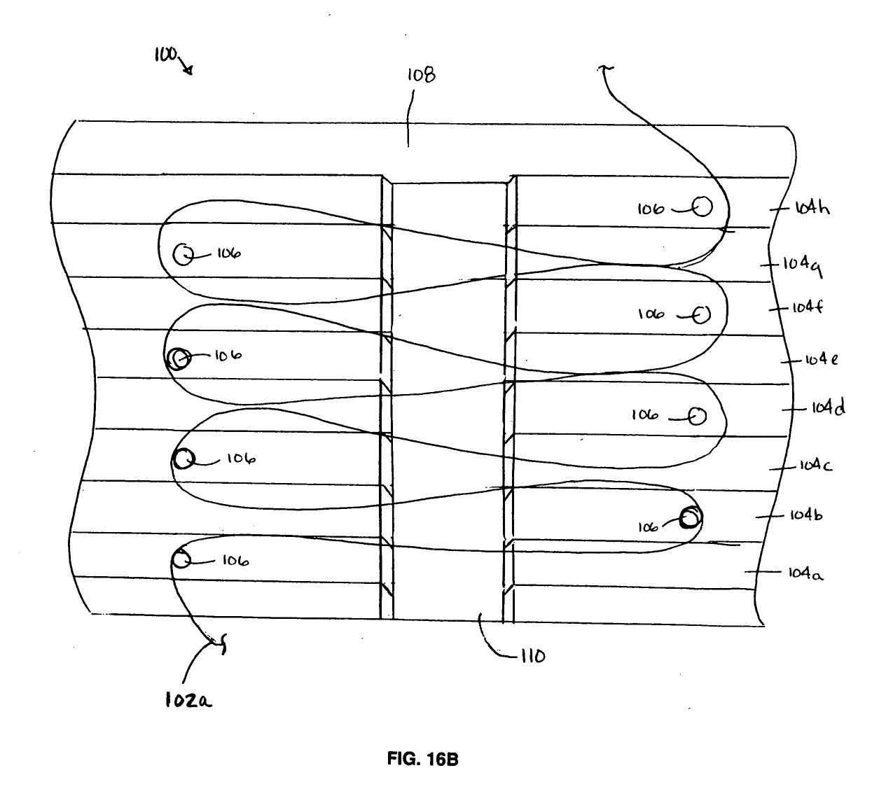

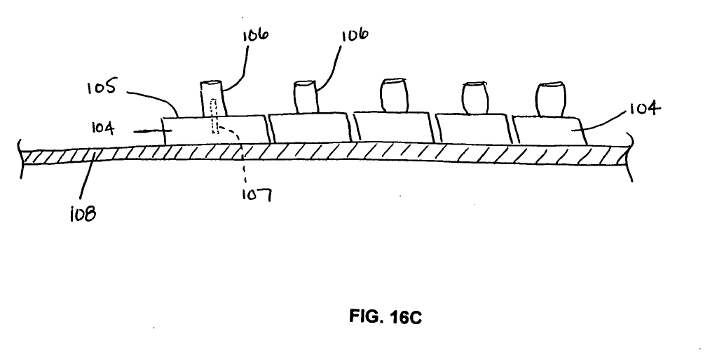

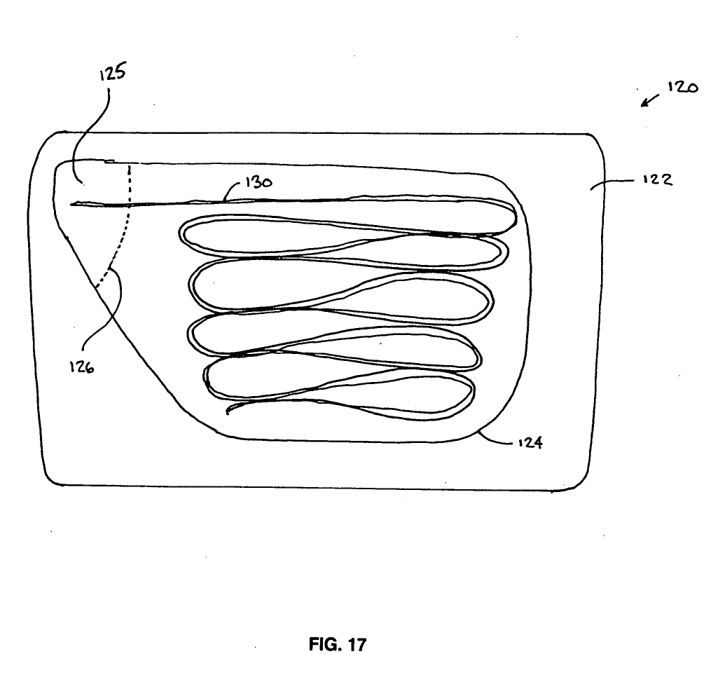

FIG. 10 illustrates the completed epidural catheter 1 placement in the patient 14 and the complete removal of the epidural needle 8 from the patient 14. As illustrated, the epidural catheter 8 has been placed in the patient 14 with the distal catheter end 19 in the patient's epidural space and the proximal catheter end 20 controlled by the physician's left hand 12 ready to receive medication. The epidural catheter has been completely extracted from the dispenser 18 and the dispenser's inner cavity 5. The epidural catheter has also completely traversed through the epidural needle's central bore. Having completed the epidural catheter placement procedure, the physician's right hand 17 continues to hold the epidural catheter dispenser system 18 in the right-hand palm and third, fourth and fifth digits 17 while also simultaneously holding the epidural needle handle 10 in the righthand thumb and index finger. The physician can now discard the dispenser and epidural needle, freeing up the right hand to administer medication through the sterile proximal epidural catheter end 20.

In combination, FIGS. 4 through 10 illustrate that the epidural catheter is placed in the patient's epidural space in a controlled and contained manner during the entire sequence of the epidural injection procedure. Because the current invention's epidural catheter dispenser system maintains the epidural catheter's sterility while containing the proximal catheter end in the dispenser's sterile inner cavity and because the only exposed portion of the epidural catheter is the distal catheter end—which the physician is controlling during the catheter's placement into the epidural needle bore—the preferred embodiment of the current invention eliminates the likelihood of catheter contamination. As the probability of catheter contamination is dramatically reduced by the current invention, correspondingly, the chance for patient infection from an epidural procedure is also dramatically reduced.

What is claimed is:

1. An epidural catheter dispenser system, the system comprising:
at least one sidewall and having a proximal end and a distal end, the distal end being connected to a distal end piece, thereby defining an inner cavity;
wherein the proximal end defines a loading aperture such that a catheter may be loaded or adjusted into the inner cavity through the loading aperture; and
wherein the distal end piece defines a dispensing aperture such that a loaded catheter in the inner cavity can be extracted from the inner cavity through the dispensing aperture.

2. The epidural catheter dispenser system of claim 1 wherein the sidewall's proximal end is further connected to a proximal end piece, thereby further defining an inner cavity,
wherein the proximal end piece defines a loading aperture such that a catheter may be loaded or adjusted into the inner cavity through the loading aperture.

3. The system of claim 1 wherein the dispenser can be no larger than a human hand.

4. The system of claim 2 wherein the dispenser can be no larger than a human hand.

5. The system of claim 1, 2, 3 or 4 wherein the dispenser is made of a semi-rigid material.

6. The system of claim 1, 2, 3 or 4 wherein the dispenser is positioned in either hand of a user such that the distal end is directed toward the user's thumb and index finger so that the catheter contained within the inner cavity may be completely extracted through the dispensing aperture;

7. The system of claim 1, 2, 3 or 4 wherein the sidewall takes the shape of a cone.

8. The system of claim 1, 2, 3 or 4 wherein the sidewall takes the shape of a cylinder.

9. The system of claim 1, 2, 3 or 4 wherein the sidewall takes the shape of a polyhedron.

10. The system of claim 1, 2, 3 or 4 wherein the inner cavity entirely confines the catheter except through the dispensing aperture.

11. The method of preventing contamination of an epidural catheter by loading a catheter in an epidural catheter dispenser system, the system comprising:
at least one sidewall, the sidewall being conical, cylindrical or polyhedral and having a proximal end and a distal end, the distal end being connected to a distal end piece, thereby defining an inner cavity;
wherein the proximal end defines a loading aperture such that a catheter may be loaded or adjusted into the inner cavity through the loading aperture, and
wherein the distal end piece defines a dispensing aperture such that a loaded catheter in the inner cavity can be extracted from the inner cavity through the dispensing aperture.

12. The method of claim 11 wherein the proximal end of the epidural catheter dispenser system's sidewall is connected to a proximal end piece, thereby further defining an inner cavity,
wherein the proximal end piece defines a loading aperture such that a catheter may be loaded or adjusted into the inner cavity through the loading aperture.

13. The method of claim 11 wherein the epidural catheter dispenser system is no larger than the human hand.

14. The method of claim 12 wherein the epidural catheter dispenser system is no larger than the human hand.

15. The method of claim 11, 12, 13 or 14 wherein the epidural catheter dispenser system is made of a semi-rigid material.

16. The method of claim 11 or 12 wherein the loading of the catheter into the epidural dispenser system is performed manually.

17. The method of claim 11 or 12 wherein the loading of the catheter into the epidural dispenser system is performed mechanically.

18. The method of claim 11 or 12 wherein the loading of the catheter into the epidural dispenser system is performed through an automated process.

19. The method of making an epidural catheter dispensing system comprising the steps of:
constructing a mold of a dispenser, the mold comprising at least a sidewall, the sidewall being conical, cylindrical or polyhedral and having a proximal end and a distal end, the distal end being connected to a distal end piece, wherein the distal end piece also has a dispensing aperture;
acquiring semi-rigid material, the semi-rigid material being of a polymer or elemental composition;
liquefying the semi-rigid material;
pouring the liquefied semi-rigid material into the mold;
solidifying the liquefied semi-rigid material in the mold; and
extracting the solidified semi-rigid material from the mold.

20. The method of claim 19 wherein the dispenser mold has additionally a proximal end piece connected to the proximal end of the sidewall, the proximal end piece defining a loading aperture.

21. The method of claim 19 or 20 wherein the liquefying step occurs through a heating or chemical process.

22. The method of claim 19 or 20 wherein the solidifying step occurs through a cooling process.

23. The method of claim 19 or 20 wherein the making of the epidural catheter dispenser system is automated.

24. The method of making an epidural catheter dispensing system comprising the steps of:
constructing a mold of a sidewall, the sidewall being conical, cylindrical or polyhedral and having a proximal and distal end;
constructing a mold of a distal end piece, allowing the distal end piece to define a dispensing aperture;
constructing a mold of a proximal end piece, allowing the proximal end piece to define a loading aperture;
acquiring semi-rigid material, the semi-rigid material being of a polymer or elemental composition;
liquefying the semi-rigid material;
pouring the liquefied semi-rigid material into each mold;
solidifying the liquefied semi-rigid material in each mold;
extracting the solidified semi-rigid material from each mold;
joining the extracted solidified semi-rigid material shapes to each other such that the sidewall connects to the proximal end piece at the proximal end and the sidewall connects to the distal end piece at the distal end.

25. The method of claim 24 wherein the making of the epidural catheter dispensing system is automated.

26. The method of claim 24 wherein the liquefying step occurs through a chemical or heating process.

27. The method of claim 24 wherein the solidifying step occurs through a cooling process.

28. The method of claim 24 wherein the joining step occurs through a chemical or mechanical process.

29. The method of making an epidural catheter dispensing system comprising the steps of:
constructing a mold of a sidewall, the sidewall being conical, cylindrical or polyhedral and having a proximal and distal end and a distal end piece, allowing the distal end piece to define a dispensing aperture;
constructing a mold of a proximal end piece, allowing the proximal end piece to define a loading aperture;
acquiring semi-rigid material, the semi-rigid material being of a polymer or elemental composition;
liquefying the semi-rigid material;
pouring the liquefied semi-rigid material into each mold;
solidifying the liquefied semi-rigid material in each mold;
extracting the solidified semi-rigid material from each mold;
joining the extracted solidified semi-rigid material shapes to each other such that the sidewall with the distal end piece connects to the proximal end piece at the sidewall's proximal end.

30. The method of claim 29 wherein the making of the epidural catheter dispensing system is automated.

31. The method of claim 29 wherein the liquefying step occurs through a chemical or heating process.

32. The method of claim 29 wherein the solidifying step occurs through a cooling process.

33. The method of claim 29 wherein the joining step occurs through a chemical or mechanical process.

34. The method of making an epidural catheter dispensing system comprising the steps of:
constructing a mold of a sidewall, the sidewall being conical, cylindrical or polyhedral and having a proximal and distal end and proximal end piece, allowing the proximal end piece to define a loading aperture;
constructing a mold a distal end piece, allowing the distal end piece to define a dispensing aperture;
acquiring semi-rigid material, the semi-rigid material being of a polymer or elemental composition;
liquefying the semi-rigid material;
pouring the liquefied semi-rigid material into each mold;
solidifying the liquefied semi-rigid material in each mold;
extracting the solidified semi-rigid material from each mold;
joining the extracted solidified semi-rigid material shapes to each other such that the sidewall with the proximal end piece connects to the distal end piece at the sidewall's distal end.

35. The method of claim 34 wherein the making of the epidural catheter dispensing system is automated.

36. The method of claim 34 wherein the liquefying step occurs through a chemical or heating process.

37. The method of claim 34 wherein the solidifying step occurs through a cooling process.

38. The method of claim 34 wherein the joining step occurs through a chemical or mechanical process.

39. The method of using an epidural catheter dispenser system to dispense an epidural catheter in an epidural injection procedure comprising the steps of:
loading the dispenser system's inner cavity with at least one catheter;
extracting one end of the catheter out of the dispenser's inner cavity through a dispenser aperture in a distal end piece;
inserting the extracted end of the catheter into the bore of an epidural needle;
advancing the catheter from the dispenser's inner cavity through the bore of an epidural needle; and
pulling the epidural needle over the entire length of the catheter as the catheter is simultaneously being extracted from the dispenser's inner cavity,
wherein the epidural catheter dispensing system comprises: at least one sidewall and having a proximal end and a distal end, the distal end being connected to a distal end piece, thereby defining an inner cavity;
wherein the proximal end defines a loading aperture such that a catheter may be loaded or adjusted into the inner cavity through the loading aperture; and
wherein the distal end piece defines a dispensing aperture such that a loaded catheter in the inner cavity can be extracted from the inner cavity through the dispensing aperture.

40. The method of claim 39 wherein the epidural catheter dispenser system further comprises a proximal end piece connected to the proximal end of the sidewall, thereby further defining an inner cavity,
and wherein the proximal end piece defines a loading aperture such that a catheter may be loaded or adjusted into the inner cavity through the loading aperture.

41. The method of claim 39 or 40 wherein the loading step is performed manually or mechanically.

42. The method of claim 39 or 40 wherein the loading step is automated.

43. The method of claim 39 or 40 wherein the extracting step is performed manually or mechanically.

44. The method of claim 39 or 40 wherein the extracting step is automated.

45. The method of claim 39 or 40 wherein the inserting step is performed with either hand of a medical practitioner.

46. The method of claim 39 or 40 wherein the inserting step is performed with both hands of a medical practitioner.

47. The method of claim 39 or 40 wherein the advancing step is performed with either hand of a medical practitioner.

48. The method of claim 39 or 40 wherein the inserting step is performed with both hands of a medical practitioner.

49. The method of claim 39 or 40 wherein the pulling step is performed with either hand of a medical practitioner.

50. The method of claim 39 or 40 wherein the pulling step is performed with both hands of a medical practitioner.
